(19) United States
(12) Patent Application Publication
(10) Pub. No.: US 2025/0251474 A1
(43) Pub. Date: Aug. 7, 2025

(54) RECEIVE COIL UNIT AND MEDICAL IMAGE DIAGNOSTIC SYSTEM

(71) Applicant: FUJIFILM Corporation, Tokyo (JP)

(72) Inventors: Yosuke OTAKE, Tokyo (JP); Hisaaki OCHI, Tokyo (JP); Koichi ARAI, Tokyo (JP)

(73) Assignee: FUJIFILM Corporation, Tokyo (JP)

(21) Appl. No.: 19/029,700

(22) Filed: Jan. 17, 2025

(30) Foreign Application Priority Data
Feb. 2, 2024 (JP) 2024-015012

(51) Int. Cl. G01R 33/34; A61B 5/055; G06T 7/00

(52) U.S. Cl. CPC G01R 33/34084; A61B 5/055; G06T 7/0012; G06T 2207/10088

(57) ABSTRACT

Provided are a receive coil unit and a medical image diagnostic system capable of reducing a burden on an imaging staff. A receive coil unit includes a band member, a first connecting tool and a second connecting tool that are provided at both ends of the band member and connect the band member to a table, a receive coil that includes a plurality of coil elements receiving a nuclear magnetic resonance signal of a subject, and a hook member that is disposed on the receive coil and holds the band member so that the receive coil is slidable along the band member.

### CROSS-REFERENCE TO RELATED APPLICATION

The present application claims priority under 35 U.S.C § 119 (a) to Japanese Patent Application No. 2024-015012 filed on Feb. 2, 2024, which is hereby expressly incorporated by reference, in its entirety, into the present application.

### BACKGROUND OF THE INVENTION

### 1. Field of the Invention

The present invention relates to a receive coil unit and a medical image diagnostic system, and relates to a technique for reducing a burden on an imaging staff.

### 2. Description of the Related Art

In a case where a subject is imaged by a magnetic resonance imaging (MRI) apparatus, the subject is disposed in an imaging space of a gantry together with a table of a bed device. In this case, in order to obtain an image of an imaging part (for example, a chest or an abdomen) of the subject, a receive coil unit such as a radio frequency (RF) coil that receives a nuclear magnetic resonance (NMR) signal is mounted on the subject.

JP2019-92935A discloses a technique for fixing a receive coil unit to a subject. According to JP2019-92935A, the receive coil is mounted on the imaging part, and then the receive coil unit is fixed to the table together with the subject by a fixing belt.

### SUMMARY OF THE INVENTION

In recent years, there has been a demand for a technique for improving a burden (workflow) on an imaging staff in a case where the receive coil unit is mounted on and fixed to the subject. In the technique of JP2019-92935A, since the receive coil unit and the fixing belt (further, the subject and the abdominal coil) are separated from each other, two work steps of mounting the receive coil unit and fixing the receive coil unit to the table by the fixing belt are required. Therefore, there is a problem that the burden on the imaging staff increases.

The present invention has been made in view of such circumstances, and an object of the present invention is to provide a receive coil unit and a medical image diagnostic system capable of reducing a burden on an imaging staff.

A first aspect relates to a receive coil unit comprises a band member, a first connecting tool and a second connecting tool that are provided at both ends of the band member and connect the band member to a table, a receive coil that includes a plurality of coil elements receiving a nuclear magnetic resonance signal of a subject, and a hook member that is disposed on the receive coil and holds the band member so that the receive coil is slidable along the band member.

A second aspect relates to the receive coil unit according to the first aspect, in which the receive coil does not have the hook members at both ends and includes weight members at the both ends.

In a third aspect, in the receive coil unit according to the first aspect, the receive coil includes a plurality of coil elements and a plurality of plate-shaped members that support the plurality of coil elements and are disposed to be spaced apart from each other, the plurality of plate-shaped members are configured to be folded, and the hook member is disposed to be spaced apart from the plurality of plate-shaped members.

In the receive coil unit of a fourth aspect, in any one of the first to third aspects, the receive coil includes a flexible coil cover that covers a periphery of the plurality of coil elements.

In the receive coil unit of a fifth aspect, in the first aspect, the receive coil includes the plurality of coil elements and a rigid body portion that holds the plurality of coil elements.

In the receive coil unit according to a sixth aspect, in any one of the first to fifth aspects, the first connecting tool includes a length adjustment mechanism of the band member and a tightening-up mechanism.

In the receive coil unit of a seventh aspect, in any one of the first to sixth aspects, the first connecting tool includes a length measurement sensor for measuring a length of the band member and/or a connection confirmation sensor for confirming presence or absence of connection of the first connecting tool, and a transmission line that transmits a detection signal from the length measurement sensor and/or the connection confirmation sensor to the receive coil.

An eighth aspect relates to the medical image diagnostic system comprises the receive coil unit according to any one of the first to seventh aspects, a table including a movable top plate on which a subject is placed, and a magnetic resonance imaging apparatus that including a processor that processes a nuclear magnetic resonance signal received by the receive coil unit.

In a ninth aspect, in the medical image diagnostic system according to the eighth aspect, the movable top plate includes a movement rail along the longitudinal direction, to which the second connecting tool is slidably connected.

In a medical image diagnostic system according to a tenth aspect, in the eighth aspect or the ninth aspect, the table includes a retracting rail for retracting the receive coil unit.

An eleventh aspect relates to the medical image diagnostic system according to any one of the eighth to tenth aspects, in which the receive coil unit includes a coil connector, and the table includes an accommodation space that accommodates the coil connector.

A medical image diagnostic system according to a twelfth aspect comprises the receive coil unit according to any one of the first to sixth aspects, a length measurement sensor for measuring a length of the band member provided in the first connecting tool, a connection confirmation sensor for confirming presence or absence of connection of the first connecting tool, and a transmission line for transmitting a detection signal from the length measurement sensor and the connection confirmation sensor to the receive coil, and a magnetic resonance imaging apparatus including a processor and an input receiving device that process a nuclear magnetic resonance signal received by the receive coil unit, in which the processor is configured to determine an SAR limit value on the basis of the detection signal from the length measurement sensor, and display the SAR limit value on the input receiving device, and the input receiving device is configured to receive a change of an imaging parameter.

According to the present invention, it is possible to reduce the burden on the imaging staff for mounting the receive coil unit.

### BRIEF DESCRIPTION OF THE DRAWINGS

FIG. 1 is an external perspective view of a medical image diagnostic system according to an embodiment.

FIG. 2 is a schematic diagram showing an internal configuration of an MRI apparatus.

FIG. 3 is an exploded perspective view schematically showing a configuration of a receive coil of the first embodiment.

FIG. 4 is a top view of the receive coil unit of the first embodiment.

FIG. 5 is a top view of a modification example of the receive coil unit of the first embodiment.

FIGS. 6A and 6B are diagrams for describing a procedure of mounting the receive coil unit of FIG. 5 on a subject.

FIGS. 7A and 7B are diagrams for describing a modification example of the receive coil unit of the first embodiment.

FIG. 8 is an exploded perspective view schematically showing a configuration of a receive coil of a second embodiment.

FIGS. 9A and 9B are diagrams for describing a receive coil unit of a second embodiment.

FIG. 10 is a diagram for describing a modification example of the receive coil unit of the second embodiment.

FIGS. 11A and 11B are diagrams for describing a procedure of mounting the receive coil unit of FIG. 10 on the subject.

FIGS. 12A and 12B are diagrams for describing a modification example of the receive coil unit according to the second embodiment.

FIG. 13 is a diagram for describing a receive coil unit of a second embodiment.

FIG. 14 is a diagram for describing a usage state of the receive coil unit of the embodiment.

FIGS. 15A and 15B are diagrams for describing another usage state of the receive coil unit of the embodiment.

FIG. 16 is a diagram for describing another usage state of the receive coil unit of the embodiment.

FIG. 17 is a diagram for describing another usage state of the receive coil unit of the embodiment.

FIG. 18 is a flowchart showing a procedure of changing the imaging parameter using the receive coil unit shown in FIG. 17.

### DESCRIPTION OF THE PREFERRED EMBODIMENTS

Hereinafter, preferred embodiments of the present invention will be described in detail with reference to the accompanying drawings. It should be noted that, in the following description and the accompanying drawings, the same reference numerals denote the same constituent elements, and the duplicated description thereof is omitted. In the following embodiments, in a case in which a plurality of constituent elements are described and listed, it can be interpreted that at least one of the plurality of constituent elements is included.

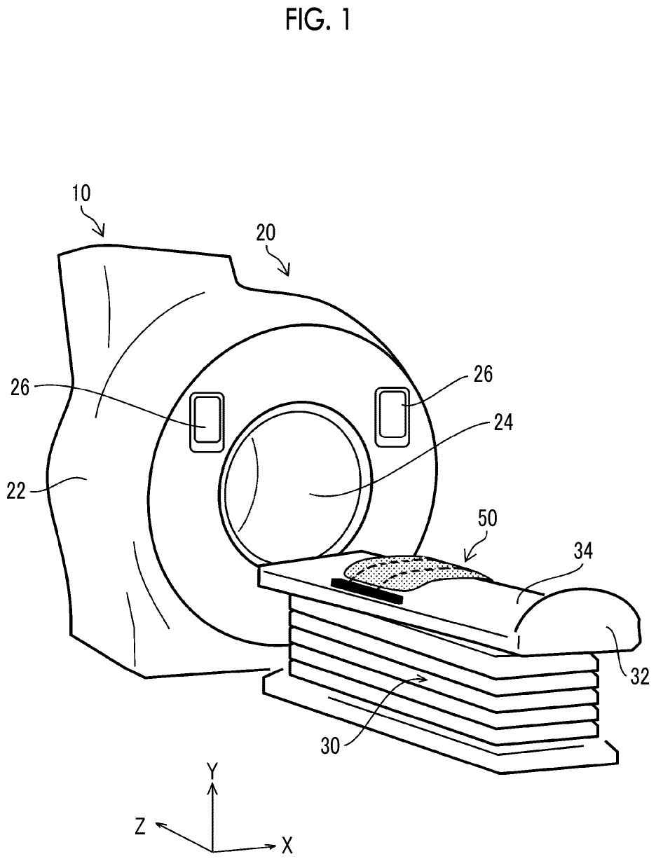

As shown in FIG. 1, a medical image diagnostic system 10 of the embodiment is a magnetic resonance imaging apparatus (hereinafter, referred to as an MRI apparatus) 20, and includes a receive coil unit 50.

### MRI Apparatus

The MRI apparatus 20 is installed in an examination room of the image diagnostic facility. In the examination room, the subject is placed on a movable top plate 34 of a table 32 of the bed device 30. Thereafter, the movable top plate 34 is moved, and the subject is transported toward the static magnetic field generating magnet 102 of the MRI apparatus 20.

The MRI apparatus 20 includes a static magnetic field generating magnet 102. The static magnetic field generating magnet 102 has a cylindrical shape, and has the imaging space 24 above the center of the cylindrical shape, and the table 32 is moved in the imaging space 24. The gantry monitor 26 is installed on both left and right sides of the static magnetic field generating magnet 102. The gantry monitor 26 also functions as an operation panel.

The three-dimensional coordinate system illustrated in FIG. 1 illustrates an example of the definition of the direction in the MRI apparatus 20. An X axis, a Y axis, and a Z axis of the three-dimensional coordinate system are merely examples and the present invention is not limited thereto. In the following description, the X axis, the Y axis, and the Z axis in the MRI apparatus 20 are defined in the same direction in any drawing. In addition, as an example of the three-dimensional coordinate system, the Z-axis direction is a static magnetic field direction and is a front-rear direction. The Y-axis direction is an up-down direction of the subject. The X-axis direction is a left-right direction of the subject.

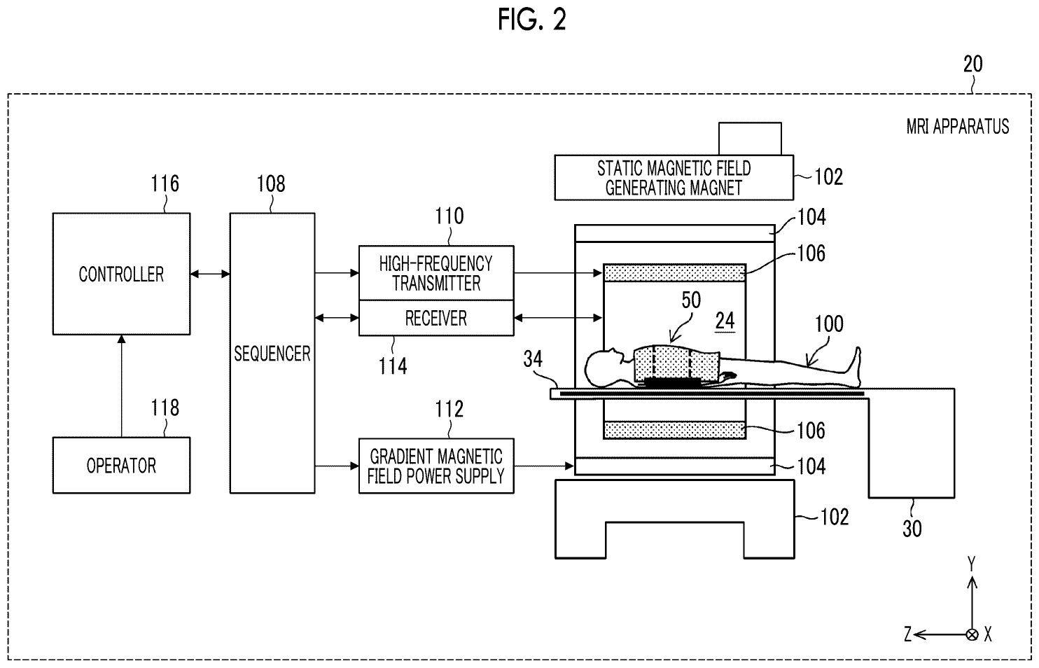

FIG. 2 is a schematic diagram showing an internal configuration of the MRI apparatus 20.

As shown in FIGS. 1 and 2, the MRI apparatus 20 includes a static magnetic field generating magnet 102, a gradient magnetic field coil 104, and a transmissive coil 106.

The static magnetic field generating magnet 102 generates a uniform static magnetic field in the imaging space 24 in which the subject 100 is disposed. The gradient magnetic field coil 104 generates a gradient magnetic field in the imaging space 24. The transmissive coil 106 generates a high-frequency magnetic field in the imaging space 24 to induce a nuclear magnetic resonance (NMR) signal (Hereinafter, the signal will be referred to as the NMR signal) in the atomic nuclei of atoms constituting the tissue of the subject 100.

The subject 100 placed on the movable top plate 34 is provided with the receive coil unit 50 mounted on the chest and abdomen of the subject 100. The table 32 on which the subject 100 is placed is moved into the imaging space 24, so that the examination site (imaging target site) of the subject 100 is positioned at the center of the static magnetic field of the imaging space 24. The receive coil unit 50 is mounted on the subject 100 and detects the NMR signal generated from the subject 100.

The sequencer 108 sends commands to the high-frequency transmitter 110 and the gradient magnetic field power supply 112 according to the imaging sequence (pulse sequence), and signals appropriately amplified by the high-frequency transmitter 110 and the gradient magnetic field power supply 112 are sent to the transmissive coil 106 or the gradient magnetic field coil 104.

The signal sent to the transmissive coil 106 is applied to the subject 100 as a pulsed high-frequency magnetic field (RF pulse) via the transmissive coil 106. The NMR signal generated from the subject 100 is detected by the coil element 52 of the receive coil unit 50, and the detection is performed by the receiver 114.

The gradient magnetic field coil 104 includes gradient magnetic field coils in the three directions of X, Y, and Z, and generates gradient magnetic fields in response to signals from the gradient magnetic field power supply 112.

A nuclear magnetic resonance frequency (detection reference frequency f0) to be used as a reference for detection in the receiver 114 is set by the sequencer 108. The sequencer 108 controls the operation of each unit according to pre-programmed timing and intensity. Among programs, a program that particularly describes the timing and intensity of RF pulses, gradient magnetic fields, and signal reception is referred to as a pulse sequence.

Various pulse sequences depending on the purpose are known, but the detailed description thereof will be omitted here.

A controller 116 controls an operation of the MRI apparatus 20 via the sequencer 108, and receives the signal detected by the receiver 114 and performs various types of signal processing, such as image reconstruction. The receiver 114 performs quadrature phase detection on the reception signal (NMR signal) which is an analog wave by the set detection reference frequency f0, performs analog-to-digital (AD) conversion, and then transmits the reception signal to the controller 116. This data is also referred to as a reception signal or measurement data.

The controller 116 receives various instruction inputs from the operator 118 and comprehensively controls each unit of the MRI apparatus 20. In addition, the controller 116 performs processing to convert the reception signal in the spatial frequency domain, received via the sequencer 108, into an image in real space by inverse Fourier transform, and generates the MRI image.

The controller 116 is implemented using a general-purpose computer, such as a personal computer or a microcomputer. The controller 116 includes a processor (for example, a central processing unit (CPU)), a read only memory (ROM), a random access memory (RAM), an input/output interface, and the like.

In the controller 116, various programs, including the control program stored in the ROM, are expanded in the RAM, and the programs expanded in the RAM are executed by the CPU. As a result, the functions of each unit of the MRI apparatus 20 are realized, and various arithmetic processing and control processing operations are executed via the input/output interface.

The operator 118 includes a mouse, a keyboard, a gantry monitor 26, and the like. The operator 118 functions as a part of a graphical user interface (GUI) that receives an input from the imaging staff. The operator 118 is an example of an input receiving device according to the embodiment of the present invention.

The imaging staff inputs the activation, stop (including pause), selection of a pulse sequence, imaging conditions, processing conditions, and the like of the MRI apparatus 20 into the operator 118.

### Receive Coil Unit of First Embodiment

The receive coil unit 50 according to the first embodiment will be described. The receive coil unit 50 has a blanket-like shape, and a thin, lightweight, and flexible structure is applied to the receive coil unit 50 as compared with the integrally molded type. The receive coil unit 50 is deformable according to the physique of the subject 100, and can image various examination sites. The receive coil unit 50 is an example of a receive coil unit according to the embodiment of the present invention.

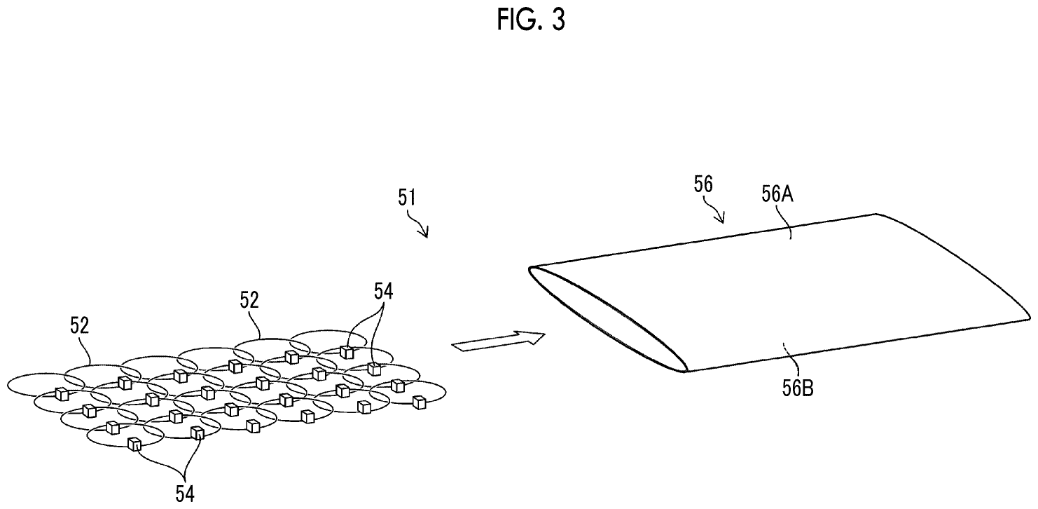

FIG. 3 is an exploded perspective view schematically showing a configuration of the receive coil 51 constituting the receive coil unit 50. As shown in FIG. 3, the receive coil 51 includes a plurality of coil elements 52, a plurality of signal processing circuits 54 that are electric components connected to each of the plurality of coil elements 52, and a coil cover 56.

The coil element 52 functions as a detector (sensor) that receives the NMR signal. The coil element 52 consists of, for example, a conductor having a diameter of about 10 cm to 15 cm and has a ring shape. The coil elements 52 are two-dimensionally arranged inside the coil cover 56. The receive coil unit 50 of the present example includes 24 coil elements 52 and is multi-channelized by being arranged in an array so as not to electromagnetically interfere with each other. The number and arrangement of the coil elements 52 are not limited to the example of FIG. 3. The coil element 52 may be configured to be deformable according to the physique of the subject 100. The coil cover 56 is an example of a coil cover according to the embodiment of the present invention.

The signal processing circuit 54 is composed of an impedance matching circuit, an amplification circuit, and the like, and for example, an electric circuit including a plurality of circuit elements may be packaged in a housing of a cube or a rectangular parallelepiped. The signal processing circuit 54 further includes a magnetic coupling prevention circuit for preventing the energy emitted from the transmissive coil 106 from entering and for removing the coupling between the coil element 52 and the transmissive coil 106. The magnetic coupling prevention circuit is configured with a capacitor, a diode, and an inductor. The inductor and the diode are connected in series to form a series circuit. This series circuit is connected in parallel with the capacitor. The diode is connected to the magnetic coupling prevention circuit driving device. In the parallel resonance circuit consisting of a capacitor, an inductor, and a diode, in a case where the diode is ON, the resonance frequency can be matched to the resonance frequency of the transmissive coil 106 which is adjusted to the same frequency as the magnetic resonance frequency. As a result, the magnetic coupling between the transmissive coil 106 and the receive coil unit 50 is prevented.

The coil cover 56 is a cover that constitutes a housing portion covering the periphery of the plurality of coil elements 52 and the signal processing circuit 54. An electric component including a plurality of coil elements 52 and a plurality of signal processing circuits 54 is accommodated inside the coil cover 56, and the blanket-shaped receive coil 51 is configured. The plurality of coil elements 52 and the plurality of signal processing circuits 54 may be accommodated in the coil cover 56 in a state of being fixed on a film (not shown). The film is a support member that fixes a positional relationship between the coil element 52 and the signal processing circuit 54 and suppresses misregistration.

The coil cover 56 is formed in a bag shape by sewing or adhering an end part of a sheet-like material cut into one piece. In the present example, the first sheet body 56A and the second sheet body 56B are sewn or adhered to each other to form the bag-shaped coil cover 56. The material of the coil cover 56 may be a urethane-based resin such as polyurethane, a polyamide synthetic resin such as nylon, or the like. A surface of the first sheet body 56A exposed to the outside constitutes an outer side surface (hereinafter, also referred to as a non-contact outer side surface) that is an outer side surface that does not come into contact with the subject. A surface of the second sheet body 56B exposed to the outside constitutes an outer side surface (hereinafter, also referred to as a contact outer side surface) that is an outer side surface in contact with the subject. A signal cable (not shown) is connected to the coil cover 56. The signal cable is a unit in which multi-channel cables for obtaining signals from each of the plurality of coil elements 52 are collected, and is electrically connected to the plurality of signal processing circuits 54.

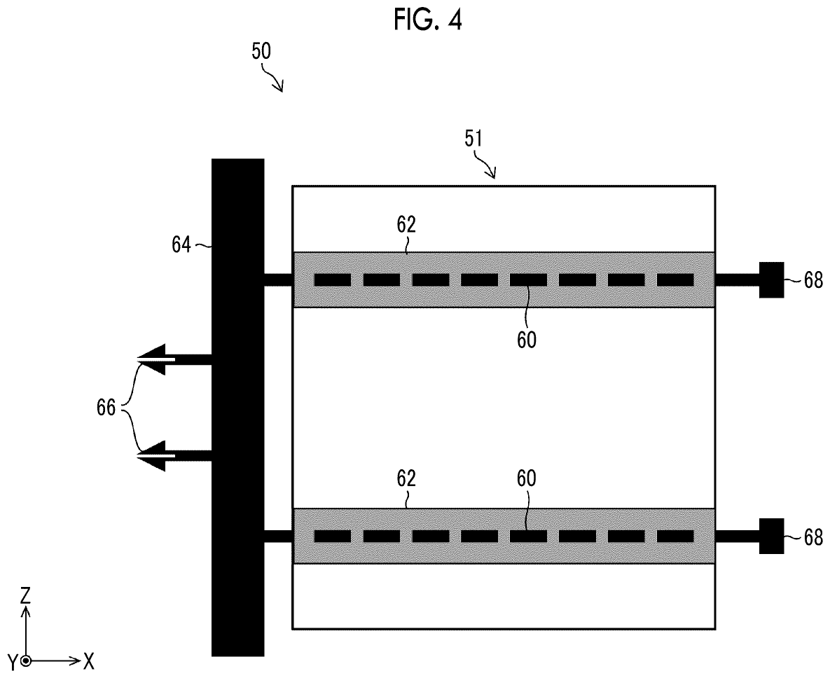

FIG. 4 is a top view of the receive coil unit 50 including the receive coil 51 as viewed from the first sheet body 56A. The receive coil unit 50 includes a receive coil 51, a guide band 60, a band hook 62, a handle 64, a buckle 66, and a rail runner 68.

As shown in FIG. 4, the guide band 60 is a string-like member having a length that exceeds both ends facing each other in the receive coil 51. In the present example, each guide band 60 extends along the X-axis direction. The two guide bands 60 are disposed side by side along the Z-axis direction at a certain distance. The number of guide bands 60 is not limited to two, and may be one or more than two. The guide band 60 may have elasticity. By having the elasticity, the subject 100 can be brought into contact with the receive coil 51 even in the subject having a different body shape. The guide band 60 is an example of a band member according to the embodiment of the present invention.

The handle 64 is provided at one end of the two guide bands 60. One handle 64 is applied as a member common to the two guide bands 60. The handle 64 is a member that is gripped by the imaging staff in a case where the guide band 60 is fixed to the movable top plate 34. The size, shape, and material of the handle 64 are not particularly limited as long as the handle 64 is easy for the imaging staff to grip.

The two buckles 66 are provided on a side of the handle 64 opposite to a side on which the guide band 60 is provided. The two buckles 66 are members for one-touch-connecting (fixing) the guide band 60 to the movable top plate 34. The buckle 66 and the movable top plate 34 are, for example, connected by snap-fitting as will be described later. The handle 64 and the buckle 66 are an example of a first connecting tool of the present invention.

The rail runner 68 is provided at each of the other ends of the two guide bands 60. The rail runner 68 is a member that is movably attached to the movable top plate 34 as will be described later. The rail runner 68 is an example of a second connecting tool according to the embodiment of the present invention.

The band hook 62 is attached to the coil cover 56. The band hook 62 is a member for inserting and holding the guide band 60. The band hook 62 of the present example is composed of a plurality of strip-shaped members extending in the Z-axis direction. The plurality of strip-shaped members are arranged to be spaced apart from each other along the X-axis direction. The guide band 60 is slidably inserted into the band hook 62. The receive coil 51, the guide band 60, and the band hook 62 are integrated to form the receive coil unit 50. The receive coil 51 can be moved (slid) in the X-axis direction along the guide band 60 by the band hook 62. Since the band hook 62 is formed of a strip-shaped member, as shown in FIG. 4, the guide band 60 has a configuration in which a portion covered with the strip-shaped member and a portion exposed to the outside appear alternately. It is preferable that the band hook 62 is made of a flexible material, as in the coil cover 56. Although an example in which the band hook 62 is formed of a plurality of strip-shaped members has been described, the configuration of the band hook 62 is not particularly limited as long as the guide band 60 can be inserted and the receive coil 51 can be moved along the guide band 60. The band hook 62 may have a tube shape (or a tubular shape) shape in which openings are formed at both ends and a space communicating with the openings is provided. As long as the shape is a tube shape (or a tubular shape), the guide band 60 can be slidably inserted. The band hook 62 is an example of a hook member of the present invention.

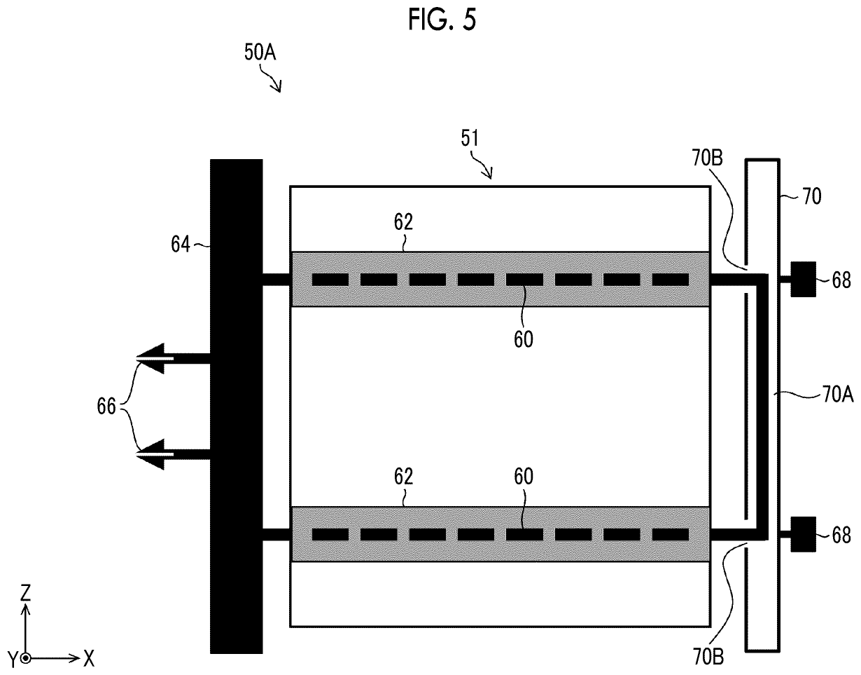

FIG. 5 is a top view of a receive coil unit 50A which is a modification example of the receive coil unit 50. In FIG. 5, the same reference numerals are assigned to the portions common to the above-described embodiment, and the description thereof will be omitted.

Unlike the receive coil unit 50, the receive coil unit 50A shown in FIG. 5 includes a threading rod 70 between the receive coil 51 and the rail runner 68. The rail runner 68 is attached to the threading rod 70.

The threading rod 70 has a hollow structure having a space 70A inside, and has two openings 70B for passing the guide band 60. In the present example, one guide band 60 is disposed to pass through the space 70A from the two openings 70B of the threading rod 70. The guide band 60 has a U-shape arrangement pattern as a whole. The size, shape, and material of the threading rod 70 are not particularly limited as long as the guide band 60 can be passed through the threading rod 70. It is preferable that the threading rod 70 is composed of a member having rigidity. By passing one guide band 60 through the threading rod 70, the threading rod 70 can define the position of the guide band 60 on the side of the rail runner 68. The threading rod 70 can function as a handle that can be gripped by the imaging staff. The imaging staff can easily fit the rail runner 68 to the movement rail of the movable top plate 34 by gripping the threading rod 70.

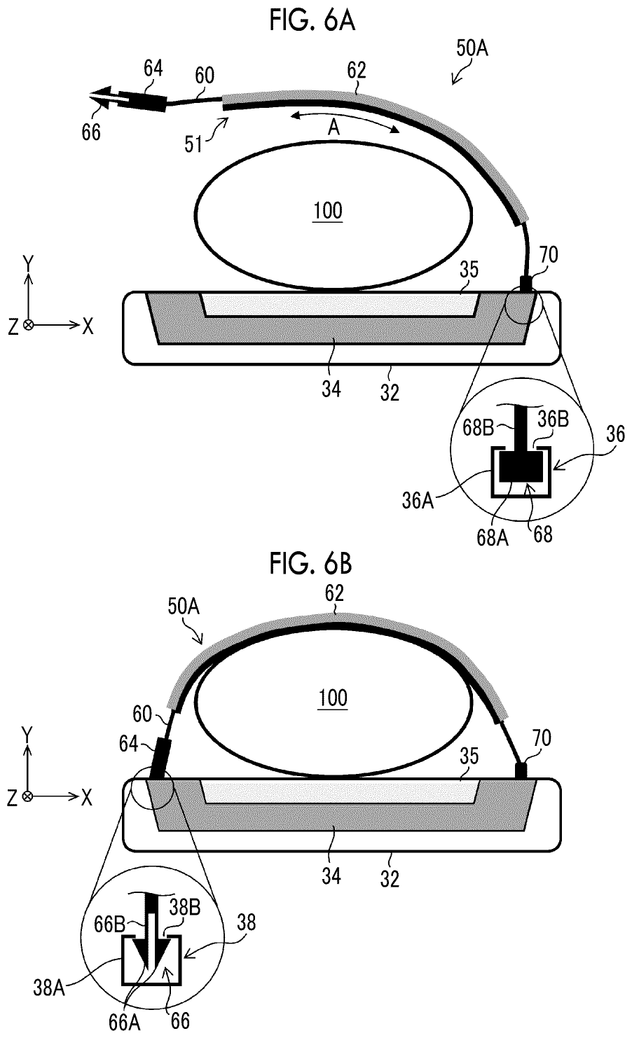

FIGS. 6A and 6B are diagrams for describing a procedure of mounting the receive coil unit 50A on the subject 100, and is a diagram viewed from the rear side to the front side in the Z-axis direction. FIG. 6A is a diagram for describing a state before the receive coil unit 50A is fixed to the movable top plate 34 by the buckle 66, and FIG. 6B is a diagram for describing a state in which the receive coil unit 50 is mounted on the subject 100 and fixed to the movable top plate 34 by the buckle 66.

As shown in FIG. 6A, the subject 100 is placed on the movable top plate 34. The rear surface coil 35 is disposed on a side of the movable top plate 34 on which the subject 100 is placed. As shown in the enlarged view, the rail runner 68 is fitted into the movement rail 36 of the movable top plate 34 and is connected to the movement rail 36. The rail runner 68 is composed of a head 68A and a neck 68B. The head 68A has a width in the X-axis direction larger than that of the neck 68B. The movement rail 36 is composed of a substantially rectangular frame-shaped member 36A in which a groove 36B is formed on the upper side. The width of the head 68A is smaller than the width of the frame-shaped member 36A and is larger than the width of the groove 36B. The width of the neck 68B is smaller than the width of the groove 36B. With this configuration, the rail runner 68 can be moved forward and backward along the Z-axis direction. On the other hand, the rail runner 68 has a so-called falling prevention structure in which the movement of the rail runner 68 in the upward direction in the Y-axis direction is restricted by the movement rail 36. The size, shape, and material of the rail runner 68 are not particularly limited as long as the rail runner 68 can be moved along the movement rail 36. The movement rail 36 is an example of a movement rail according to the embodiment of the present invention.

The imaging staff holds the handle 64, wraps the receive coil 51 around the subject 100, and brings the receive coil 51 into close contact with the observation site of the subject 100. In this case, in the receive coil unit 50A of the present example, the receive coil 51 can be moved along the guide band 60 in a direction indicated by an arrow A. The imaging staff can easily move the receive coil 51 to the observation site of the subject 100.

Next, as shown in FIG. 6B, the imaging staff can hold the handle 64 and insert the buckle 66 into the fixed rail 38 provided on the movable top plate 34. Accordingly, the buckle 66 and the fixed rail 38 are connected (fixed) to each other. The buckle 66 is composed of a claw portion (protruding portion) 66A and an elastic member 66B. The distance between the two claw portions 66A can be freely changed by the elastic force of the elastic member 66B. The fixed rail 38 is composed of a substantially rectangular frame-shaped member 38A in which a groove 38B is formed on the upper side. In a case where the buckle 66 is connected (fixed) to the fixed rail 38, the distance between the two claw portions 66A is reduced by applying a force to the elastic member 66B. The claw portion 66A can pass through the groove 38B in the downward direction. In a case where the claw portion 66A passes through the groove 38B, the claw portion 66A returns to the initial position due to the elastic force of the elastic member 66B. As shown in FIG. 6B, the two claw portions 66A and the frame-shaped member 38A are engaged with each other, and the buckle 66 and the fixed rail 38 are connected (fixed) to each other. In a case where the fixation is released, the distance between the two claw portions 66A is reduced by applying a force to the elastic member 66B. The claw portion 66A can pass through the groove 38B in the upward direction. Accordingly, the fixation can be released. The buckle 66 and the fixed rail 38 can be easily fixed to and released from each other. The size, shape, and material of the buckle 66 are not particularly limited as long as the buckle 66 can be fixed to and released from the fixed rail of the movable top plate 34.

As shown in FIGS. 6A and 6B, the operation staff can mount the receive coil 51 on the imaging part of the subject 100 and fix the receive coil unit 50A and the subject 100 to the table 32 by performing one operation. That is, the coil and the subject can be fixed in one operation.

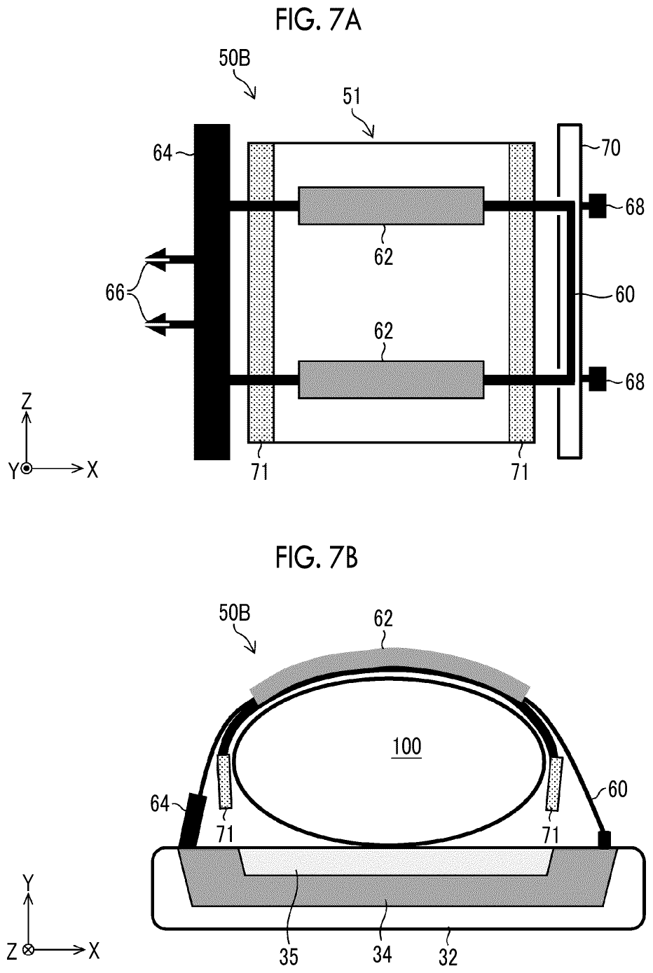

FIGS. 7A and 7B are diagrams for describing a receive coil unit 50B as a modification example of the receive coil unit 50A. In FIGS. 7A and 7B, the same reference numerals are assigned to the portions common to the above-described receive coil unit 50A, and the description thereof will be omitted. FIG. 7A is a top view of the receive coil unit 50B, and FIG. 7B is a diagram in a state in which the receive coil unit 50B is mounted on the subject 100 and fixed to the movable top plate 34.

The receive coil unit 50B shown in FIG. 7A is different from the receive coil unit 50A in that a weight 71 is provided at both ends of the receive coil 51 (a side on which the handle 64 and the rail runner 68 are provided). The receive coil unit 50B does not includes the band hook 62 at the position where the weight 71 is disposed. The band hook 62 extends from the center of the receive coil 51 to the both ends of the receive coil 51, but does not reach to the both ends. The band hook 62 of the present example is tubular, and the guide band 60 is slidably inserted into the band hook 62. The receive coil 51 can be moved along the guide band 60.

As shown in FIG. 7B, the receive coil unit 50B is mounted and fixed to the subject 100. Both ends of the receive coil 51 do not includes the band hook 62. As compared with FIG. 6B, both ends of the receive coil 51 can be moved to positions away from the guide band 60. Further, a force in the direction of gravitational force on both ends of the receive coil 51 of the weight 71. As a result, the receive coil 51 can be brought close to the side part (side waist) of the subject 100, and the image quality of the image acquired on the basis of the signal from the receive coil 51 is improved. The weight 71 is an example of a weight member according to the embodiment of the present invention. The weight may be the coil element 52 or the signal processing circuit 54. The region of the weight 71 in FIGS. 7A and 7B may have a structure in which a force of gravity acts on the region and the region hangs down more than other portions. The receive coil units 50A and 50B are examples of a receive coil unit according to the embodiment of the present invention.

### Receive Coil Unit of Second Embodiment

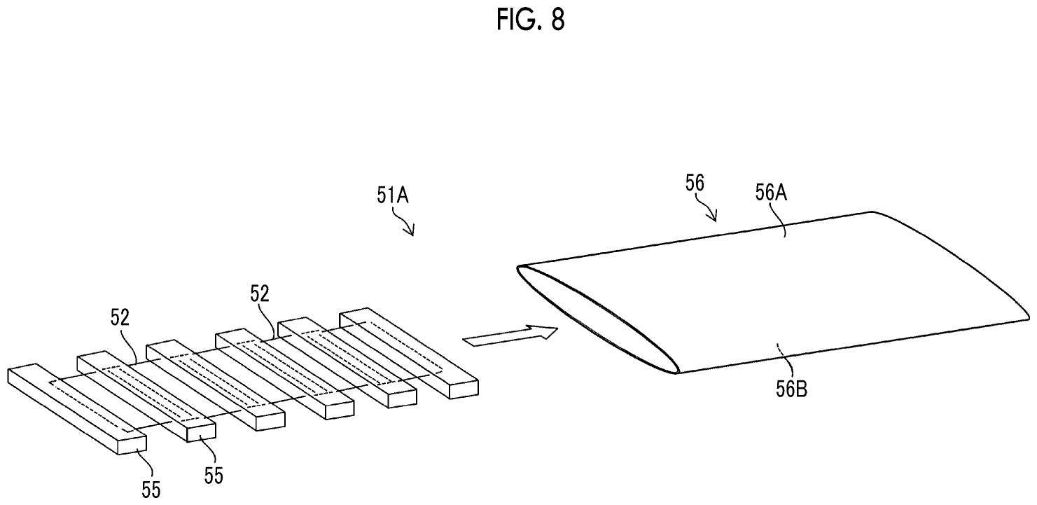

Next, the foldable receive coil unit 50C will be described. FIG. 8 shows a receive coil 51A applied to the receive coil unit 50C. The configuration of the receive coil 51A is different from the receive coil 51 of the first embodiment. As shown in FIG. 8, the receive coil 51A includes a plurality of coil elements 52 and a plurality of plate-shaped members 55 disposed to be spaced apart from each other. One coil element 52 is supported by two plate-shaped members 55. In this example, there are six plate-shaped members 55, and five coil elements 52 are supported. The plurality of coil elements 52 and the plurality of plate-shaped members 55 are accommodated in the coil cover 56. Each coil element 52 is deformable. The interval between the adjacent plate-shaped members 55 can be changed while the coil element 52 is deformed.

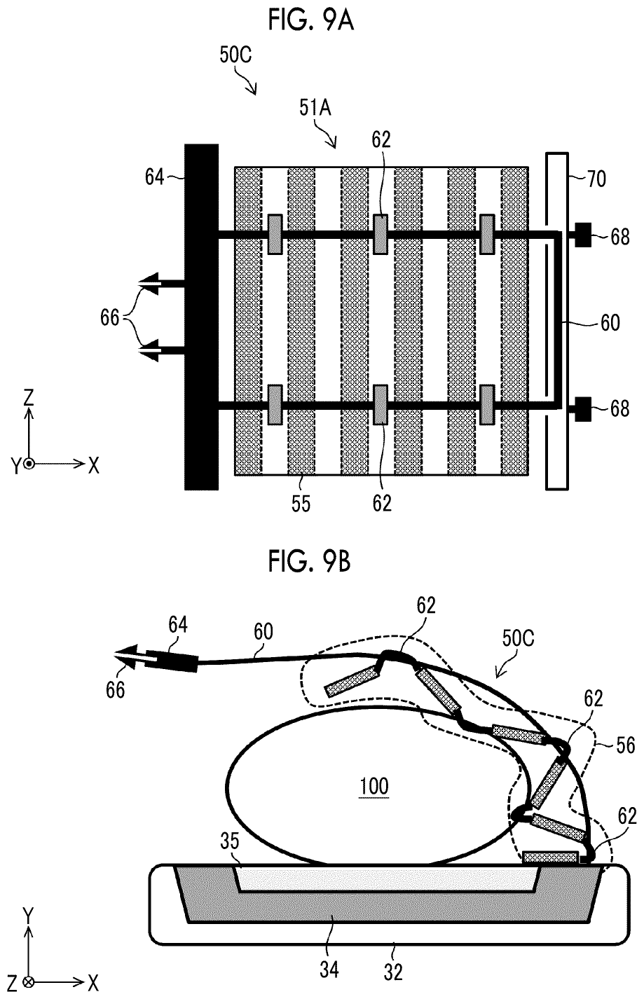

FIGS. 9A and 9B are diagrams for describing a receive coil unit of the second embodiment. FIG. 9A is a top view of the receive coil unit 50C, and FIG. 9B is a diagram of a state before the receive coil unit 50C is mounted on the subject 100, and is a diagram viewed from the rear side to the front side along the Z-axis direction. As shown in FIG. 9A, a band hook 62 is provided on the receive coil 51A. In the present example, the band hook 62 is provided in a region (non-disposed region) other than the plate-shaped member 55, that is, in a region where the plate-shaped member 55 is not disposed. However, the band hook 62 is provided in three non-disposed regions among the five non-disposed regions. The guide band 60 is slidably inserted into three band hooks 62.

As shown in FIG. 9B, the receive coil 51A includes a plurality of plate-shaped members 55 having an interval therebetween, and is configured to be foldable by changing the interval. The guide band 60 is inserted into three band hooks 62. With this configuration, the receive coil 51A can be moved along the guide band 60. In the present example, the guide band 60 passes through the inside of the coil cover 56 at a position where the guide band 60 is inserted into the band hook 62. The guide band 60 is exposed from the coil cover 56 at a position where the guide band 60 is not inserted into the band hook 62.

The imaging staff can easily register the receive coil 51 with the observation site of the subject 100 by moving the receive coil 51A before connecting the buckle 66 to the movable top plate 34. By connecting (fixing) the buckle 66 to the movable top plate 34, the receive coil unit 50A and the subject 100 can be fixed to the movable top plate 34 (table 32). That is, the coil and the subject can be fixed in one operation.

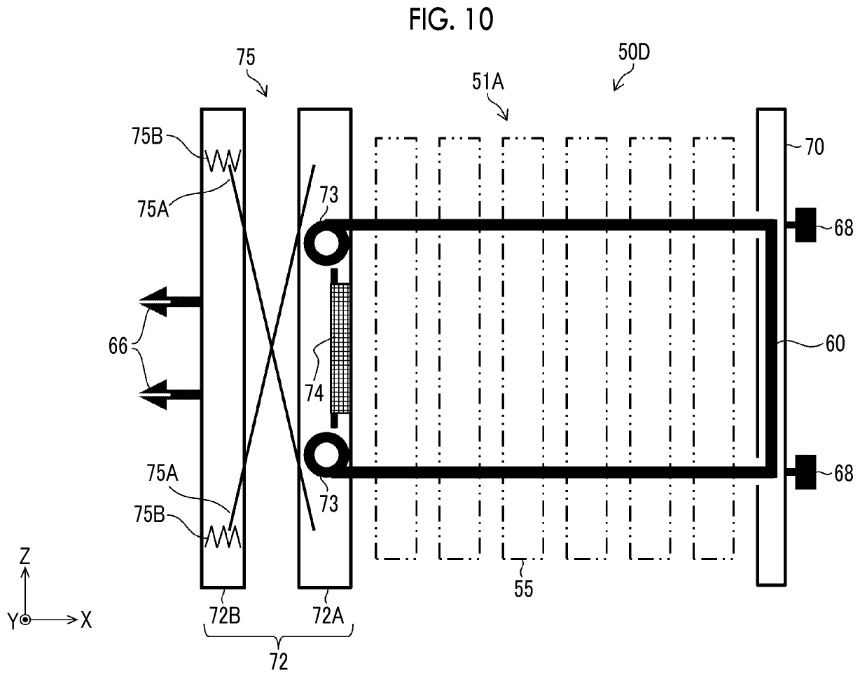

FIG. 10 is a diagram for describing a modification example of the foldable receive coil unit 50C. In FIG. 10, the same reference numerals are assigned to the portions common to the above-described receive coil unit 50C, and the description thereof will be omitted. The receive coil unit 50D shown in FIG. 10 is different from the receive coil unit 50C in that an automatic winding reel 73, a stop lever 74, and a ratchet 75 are provided on the handle 72.

The handle 72 is composed of a first handle 72A to which the guide band 60 is connected and a second handle 72B to which the buckle 66 is attached. The first handle 72A has a hollow structure having a space inside, and two automatic winding reels 73 are disposed therein. The end part of the guide band 60 is connected to each of the two automatic winding reels 73. For example, a spring type structure using a leaf spring can be applied to the automatic winding reel 73. It is preferable that the guide band 60 is made of a flexible material that does not have elasticity. Therefore, it is preferable that the length of the guide band 60 itself does not change in normal use of the guide band 60. Further, since the guide band 60 is wound around the automatic winding reel 73 by the force of the leaf spring in the initial state, the length of the guide band 60 is short. In the present example, two automatic winding reels 73 are disposed, but only one automatic winding reel 73 may be used. However, by providing two automatic winding reels 73, it is possible to prevent the guide band 60 from sliding on the subject 100. Friction between the guide band 60 and the subject 100 can be avoided.

A stop lever 74 is disposed between the two automatic winding reels 73. The stop lever 74 is a member that restricts a force of the automatic winding reel 73 in the winding direction. In a case where the stop lever 74 is operated, the winding of the automatic winding reel 73 is restricted, and the length of the guide band 60 is determined. The imaging staff can adjust the length of the guide band 60 (the length of the guide band 60 exposed from the handle 72) according to the physique of the subject 100 by the automatic winding reel 73 and the stop lever 74. The automatic winding reel 73 and the stop lever 74 are examples of a length adjustment mechanism of the present invention. In FIG. 10, the receive coil 51A does not includes the coil cover 56.

A ratchet 75 is provided on the handle 72. The ratchet 75 is composed of two claws 75A and two gears 75B. The two claws 75A are provided at positions where the first handle 72A and the second handle 72B are connected. The two claws 75A are fixed by a pin (not shown) connecting the centers of the claws 75A. The two claws 75A can be opened and closed with a pin as a fulcrum. The second handle 72B has a middle structure having a space inside and includes two linear rack type gears 75B. The two gears 75B are disposed to face each other. The distance between the first handle 72A and the second handle 72B is determined by engaging the two claws 75A with the two gears 75B. That is, the distance between the first handle 72A and the second handle 72B can be adjusted by the ratchet 75. The ratchet 75 is an example of a tightening-up mechanism of the present invention.

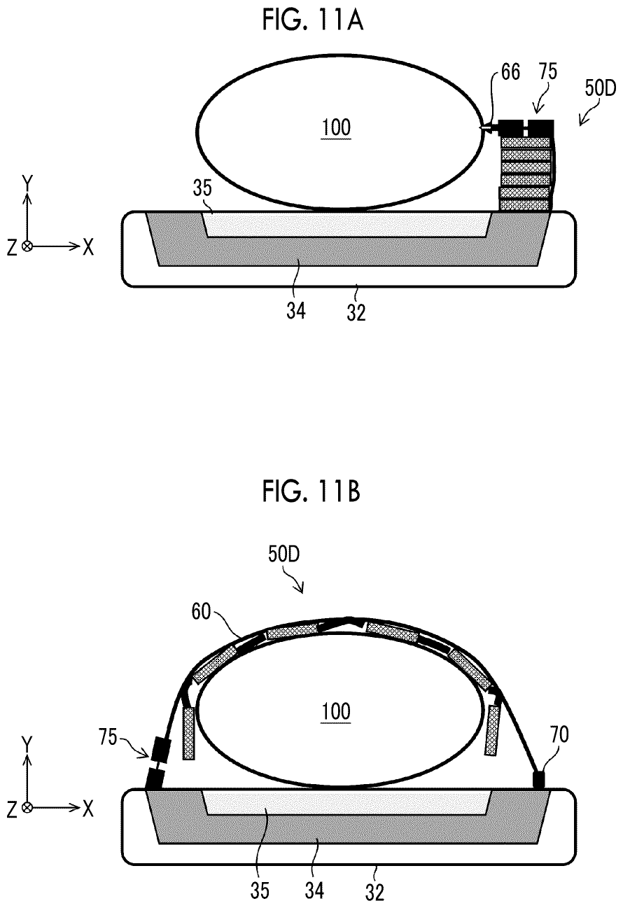

FIGS. 11A and 11B are diagrams for describing a procedure of mounting the receive coil unit 50D on the subject 100, and is a diagram viewed from the rear side to the front side in the Z-axis direction. FIG. 11A is a diagram for describing a state before the receive coil unit 50D is fixed to the movable top plate 34 by the buckle 66, and FIG. 11B is a diagram for describing a state in which the receive coil unit 50D is mounted on the subject 100 and fixed to the movable top plate 34 by the buckle 66.

As shown in FIG. 11A, the subject 100 is placed on the movable top plate 34. A rail runner 68 (not shown) of the receive coil unit 50D is fitted into a movement rail 36 (not shown) of the movable top plate 34 and is connected to the movement rail 36. The receive coil unit 50D is prepared on the movable top plate 34. In this state, the length of the guide band 60 of the receive coil unit 50D is wound by the automatic winding reel 73 (not shown), and thus the guide band 60 is in the shortest state.

As shown in FIG. 11B, the imaging staff feeds out the guide band 60 from the automatic winding reel 73 according to the physique of the subject 100. The imaging staff operates the stop lever 74 to determine the length of the guide band 60. The imaging staff holds the handle 72 and wraps the subject 100 around while moving the receive coil 51A along the guide band 60. The imaging staff makes the receive coil 51A closely contact the observation site of the subject 100. The imaging staff holds the handle 72 and inserts the buckle 66 into the fixed rail 38 provided on the movable top plate 34. Accordingly, the buckle 66 and the fixed rail 38 are connected (fixed) to each other. Further, the imaging staff operates the ratchet 75 of the handle 72 to shorten the distance between the first handle 72A and the second handle 72B. As a result, the guide band 60 can be tightened up, and the optimum fixation strength for the coil fixation and the subject fixation can be obtained. Since the handle 72 includes the automatic winding reel 73, the stop lever 74, and the ratchet 75, it is possible to achieve an optimum fixation strength even for the subject 100 of any physique. Further, since the receive coil 51A includes the plate-shaped members 55 at both ends, the receive coil 51A functions as a weight. As a result, the receive coil 51A can be brought close to the side part (side waist) of the subject 100, and the image quality of the image can be improved.

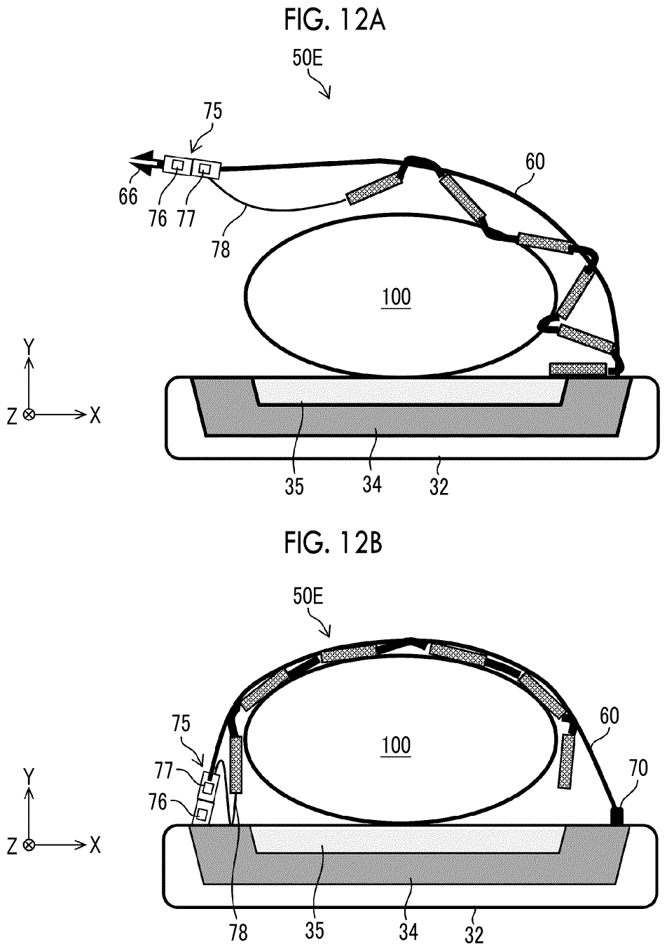

FIGS. 12A and 12B are diagrams for describing a modification example of the receive coil unit 50D. In FIGS. 12A and 12B, the same reference numerals are assigned to the portions common to the above-described receive coil unit 50D, and the description thereof will be omitted. FIG. 12A is a diagram showing a state before the receive coil unit 50E is fixed to the movable top plate 34 by the buckle 66, and FIG. 12B is a diagram showing a state in which the receive coil unit 50E is mounted on the subject 100 and fixed to the movable top plate 34 by the buckle 66.

The receive coil unit 50E shown in FIGS. 12A and 12B includes a mounting sensor 76, a length sensor 77, and a transmission line 78 on the handle 72, unlike the receive coil unit 50D.

The mounting sensor 76 is, for example, a sensor that outputs a detection signal such as ON or High in a case where the buckle 66 is inserted into the fixed rail 38, and outputs a detection signal such as OFF or Low in a case where the buckle 66 is not inserted into the fixed rail 38. The type of the sensor and the like of the mounting sensor 76 are not limited as long as the mounting sensor 76 can output a binary detection signal that can be distinguished according to the presence or absence of the insertion of the buckle 66 into the fixed rail 38. The mounting sensor 76 is an example of a connection confirmation sensor according to the embodiment of the present invention.

The length sensor 77 outputs, for example, a detection signal corresponding to the length of the guide band 60 fed out from the automatic winding reel 73. Examples of the length sensor 77 include a sensor that detects the rotation of the automatic winding reel 73 by an encoder or the like and outputs a value obtained by converting the rotation into a feed-out length as a detection signal. The type of the length sensor 77 is not limited as long as the length sensor 77 can output a detection signal corresponding to the length of the guide band 60. The length sensor 77 is an example of a length measurement sensor according to the embodiment of the present invention.

Further, a transmission line 78 connected to the mounting sensor 76 and the length sensor 77 is provided. The transmission line 78 transmits the detection signals from the mounting sensor 76 and the length sensor 77 to the receive coil 51A. The transmission line 78 is an example of a transmission line according to the embodiment of the present invention.

As shown in FIG. 12A, the imaging staff feeds out the guide band 60 from the automatic winding reel 73 according to the physique of the subject 100. The imaging staff operates the stop lever 74 to determine the length of the guide band 60. The length sensor 77 outputs a detection signal corresponding to the length of the guide band 60 fed out. The detection signal is stored in, for example, a memory.

As shown in FIG. 12B, the imaging staff holds the handle 72 and wraps the receive coil 51A around the subject 100 while moving the receive coil 51A along the guide band 60. The imaging staff makes the receive coil 51A closely contact the observation site of the subject 100. The imaging staff holds the handle 72 and inserts the buckle 66 into the fixed rail 38 provided on the movable top plate 34. In a case where the buckle 66 is inserted into the fixed rail 38, the mounting sensor 76 outputs an ON detection signal.

For example, in a case where the mounting sensor 76 outputs a detection signal in an ON state, the detection signal from the mounting sensor 76 and the detection signal from the length sensor 77 are transmitted to the receive coil 51A through the transmission line 78. As a result, the detection signal (the length information of the guide band 60 or the fixing information of the buckle 66) can be transmitted to the MRI apparatus 20 through the receive coil 51A.

In the present embodiment, one coil element 52 is supported by two plate-shaped members 55, but the present invention is not limited thereto. A structure in which two coil elements are arranged on an array and supported by two plate-shaped members 55 may be adopted. As a result, the number of coil elements 52 can be increased in the axial direction of the plate-shaped member 55, and thus the sensitivity is increased.

### Receive Coil Unit of Third Embodiment

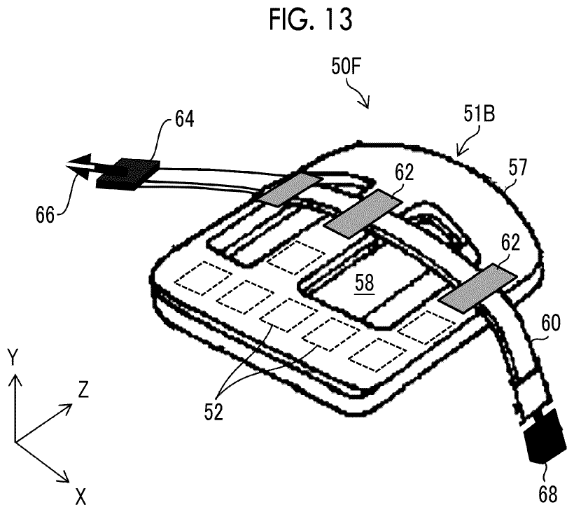

Next, the receive coil unit 50F having a curvature radius of a certain size according to the physique of the subject 100 will be described. FIG. 13 shows a receive coil 51B applied to the receive coil unit 50F. The configuration of the receive coil 51B is different from the receive coil 51 of the first embodiment and the receive coil 51A of the second embodiment.

The receive coil 51B includes a body portion 57 having a curved surface bent along the body shape of the subject 100 and a coil element 52 provided inside the body portion 57. The body portion 57 has rigidity that can maintain the shape in a normal usage state. The body portion 57 is configured such that a cutout hole 58 is provided in a portion where the coil element 52 is not present. By providing the cutout hole 58, it is possible to reduce the weight of the entire receive coil 51B. Since the body portion 57 has a gentle curved surface that is convex upward with respect to the subject 100, the body portion 57 can be mounted on the subject 100 (not shown) with good adhesiveness.

Three band hooks 62 are provided on the upper surface side of the body portion 57 of the receive coil 51B in the X-axis direction. The guide band 60 is slidably inserted into the band hook 62. As a result, the receive coil 51B can be moved along the guide band 60 in the X-axis direction.

A rail runner 68 is provided at one end of the guide band 60. A handle 64 and a buckle 66 are provided at the other end of the guide band 60.

The imaging staff fits the rail runner 68 into the movement rail 36 (not shown). The imaging staff brings the receive coil 51B into close contact with the subject 100 while moving the receive coil 51B along the guide band 60, and then holds the handle 64 to connect the buckle 66 to the fixed rail 38 (not shown). As a result, the imaging staff can perform the coil fixation and the subject fixation in one operation.

### Usage State of Receive Coil Unit of Embodiment

Next, a usage state of the receive coil unit of the embodiment will be described.

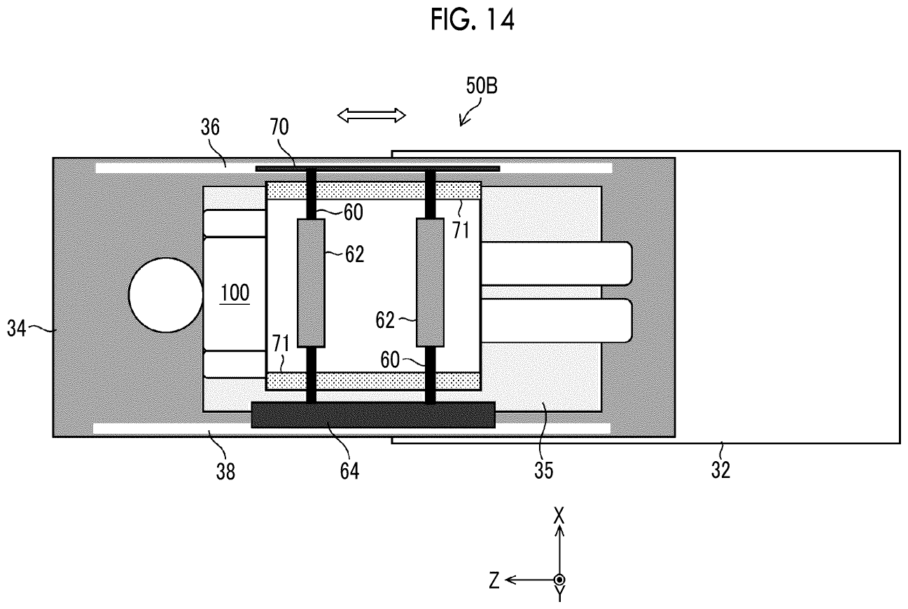

FIG. 14 is a diagram for describing a case in which the receive coil unit 50A is used, and is a top view of the subject 100 placed on the movable top plate 34. The receive coil unit 50A is mounted on the abdomen of the subject 100 shown in FIG. 14 and is connected (fixed) to the fixed rail 38 by a buckle 66.

As shown in FIG. 14, a movement rail 36 is provided on the movable top plate 34 along the longitudinal direction (Z-axis direction). The rail runner 68 (not shown) is fitted to the movement rail 36 so as to be movable. With this configuration, the receive coil unit 50A can move in the front-rear direction (Z-axis direction) as indicated by the arrow, that is, from the lower abdomen to the upper abdomen.

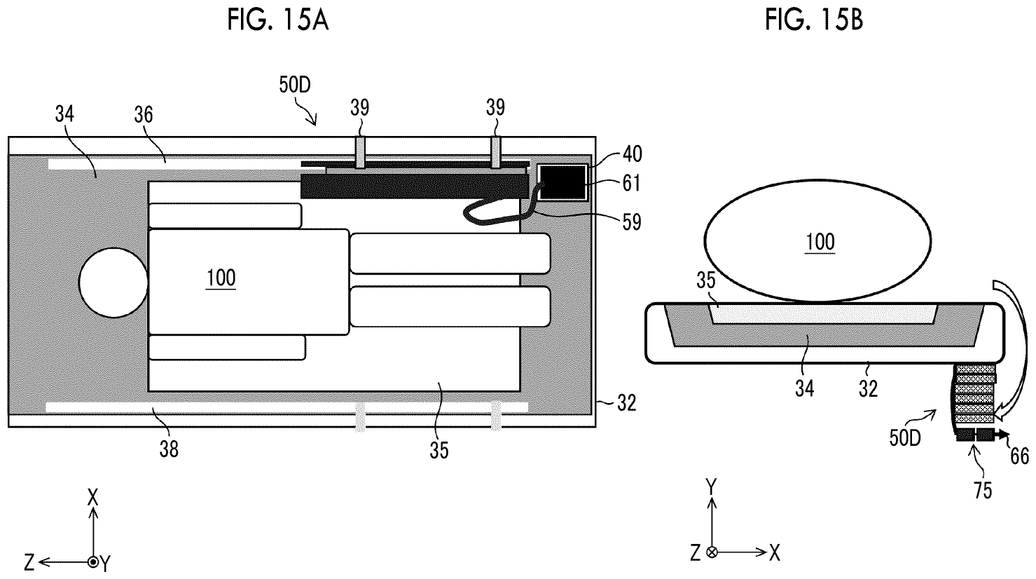

FIGS. 15A and 15B are diagrams for describing a case in which the receive coil unit 50C is used. FIG. 15A is a top view of the subject 100 placed on the movable top plate 34. FIG. 15B is a view of the subject 100 placed on the movable top plate 34 as viewed from the front side from the rear side along the Z-axis direction.

As shown in FIG. 15A, the subject 100 is placed on the movable top plate 34. The receive coil unit 50D is placed on the movable top plate 34. One end of the reception cable 59 is connected to the receive coil 51A. A coil connector 61 is connected to the other end of the reception cable 59. The coil connector 61 is connected to the connection connector 40 disposed on the movable top plate 34. The receive coil unit 50D is in a state in which the length of the guide band 60 is short due to the automatic winding reel 73. The table 32 is provided with a coil retracting rail 39. The coil retracting rail 39 is a member that retracts the coil in a case in which the receive coil unit 50C is not in use. The coil retracting rail 39 is an example of a retracting rail according to the embodiment of the present invention.

As shown in FIG. 15B, the receive coil unit 50D is guided to the coil retracting rail 39 and can be moved to the rear surface side of the table 32. As a result, since the receive coil unit 50D is removed from the movable top plate 34, the imaging staff can perform other work without being disturbed by the receive coil unit 50D. The imaging staff can perform various types of work without burden.

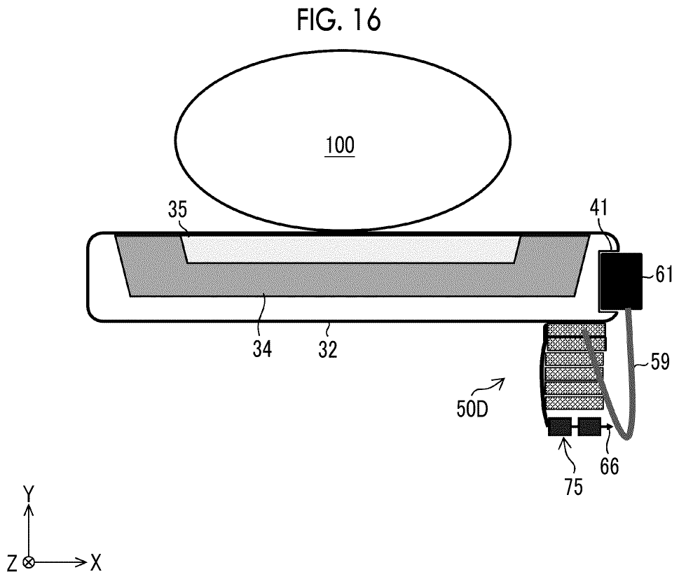

FIG. 16 is a diagram for describing a case in which the receive coil unit 50D is used. The table 32 of FIG. 16 is different from the table 32 in FIG. 15B. As shown in FIG. 16, a space 41 is provided in the table 32. The space 41 can accommodate the coil connector 61 in a case where the coil connector 61 is not in use. The table 32 can also serve as a storage place for the coil connector 61. The coil connector 61 is an example of a coil connector according to the embodiment of the present invention. The space 41 is an example of an accommodation space according to the embodiment of the present invention.

By the way, in the MRI apparatus 20, since the RF signal is emitted to the subject on which the receive coil is mounted, heat generation may occur in a part of the circuit of the receive coil that has received the irradiation. It is preferable that the RF signal to be emitted is controlled as necessary to control the heat generation of the circuit in order to prevent the damage of the receive coil. In the present embodiment, as one of the means, a function (SAR management function) of the MRI apparatus 20 of suppressing a specific absorption ratio (SAR), which is a human body absorption ratio (specific absorption ratio) of the RF signal, to a predetermined threshold value or less is utilized.

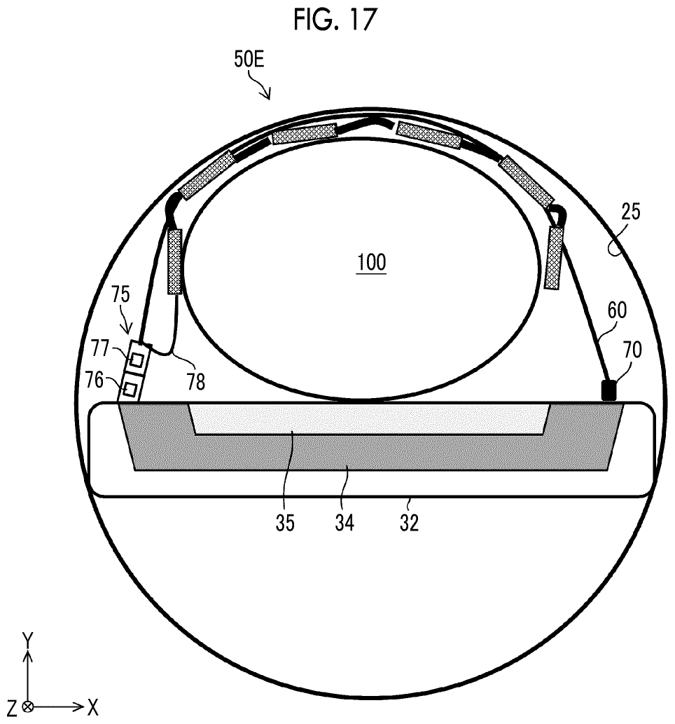

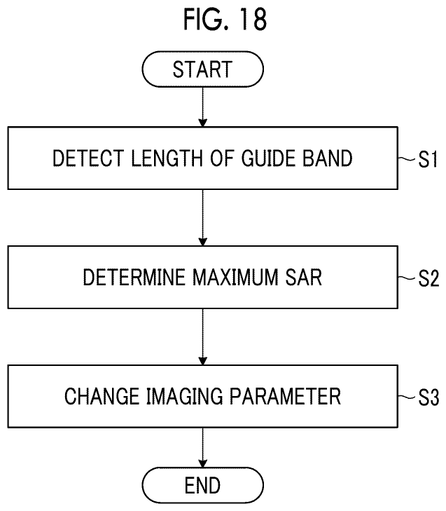

FIG. 17 is a diagram for describing a case where SAR management is performed by using the receive coil unit 50E. FIG. 18 is a flowchart showing a procedure of changing the imaging parameter.

One of the causes of the heat generation in the receive coil 51A is that the receive coil 51A approaches the interior wall 25 of the imaging space 24 of the MRI apparatus 20. A transmissive coil 106 that outputs a radio wave of high power is disposed around the interior wall 25. In a case where the receive coil 51A is located at a place close to the interior wall 25, there is a concern that the receive coil 51A receives a large power and generates heat. In order to avoid this, it is effective to suppress the output of the transmissive coil 106. On the other hand, it is difficult to know how far the receive coil 51A is located from the interior wall 25.

In the present example, the SAR management is executed by the following receive coil unit 50E and the following procedure. As described above, the receive coil unit 50E includes the length sensor 77 that measures the length of the guide band 60 and the transmission line 78 in the handle 72.

As shown in FIG. 18, in the length detection of the guide band (Step S1), the length of the guide band 60 is detected by the detection signal from the length sensor 77. Specifically, the detection signal (detection signal corresponding to the length of the guide band 60) detected by the length sensor 77 is transmitted from the transmission line 78 to the receive coil 51A and is transmitted from the receive coil 51A to the controller 116 of the MRI apparatus 20. The controller 116 detects (acquires) the length of the guide band 60 from the detection signal detected by the length sensor 77.

Next, in the determination of the maximum SAR (Step S2), the controller 116 determines the maximum SAR from the length of the guide band 60.

There is a certain relationship between the length of the guide band 60 and the physique of the subject 100. In a case where the guide band 60 is long, the size of the subject 100 is large, and in a case where the guide band 60 is short, the size of the subject 100 is small. The distance between the subject 100 and the interior wall 25 can be estimated from the length of the guide band 60. It can be estimated that the distance between the subject 100 and the interior wall 25 is short in a case where the guide band 60 is long, and the distance between the subject 100 and the interior wall 25 is long in a case where the guide band 60 is short.

The controller 116 stores a data table shown in Table 1 in advance. Here, the data table is a table in which the length of the guide band 60 and the SAR limit value are associated with each other. As shown in Table 1, the SAR limit value is 2.0 W/kg in a case where the length of the guide band 60 is 10 to 80 cm, the SAR limit value is 1.5 W/kg in a case where the length of the guide band 60 is 81 to 100 cm, and the SAR limit value is 1.0 W/kg in a case where the length of the guide band 60 is 101 to 130 cm. From the relationship between the length of the guide band 60 and the SAR limit value, it can be understood that the SAR limit value is smaller as the guide band 60 is longer. As described above, as the guide band 60 becomes longer, the subject 100 becomes closer to the interior wall 25. Therefore, the SAR limit value is set to be small in consideration of safety. The data table shown in Table 1 is an example of a combination of the length of the guide band 60 and the SAR limit value, and is not limited thereto.

TABLE 1

| Length of guide band | SAR limit value |
|---|---|
| 10 to 80 (cm) | 2.0 W/kg |
| 81 to 100 (cm) | 1.5 W/kg |
| 101 to 130 (cm) | 1.0 W/kg |

The controller 116 determines the SAR limit value from the detected length of the guide band 60 based on the data table. For example, in a case where the controller 116 detects that the length of the guide band 60 is 90 cm, the SAR limit value is determined to be 1.5 W/kg based on the data table.

Next, in the imaging parameter change (Step S3), the controller 116 displays the detected length of the guide band 60 and the determined SAR limit value on the operator 118. The imaging staff checks the content displayed on the operator 118, decides the imaging parameter, and inputs the instruction to the operator 118. Based on the instruction from the operator 118, the controller 116 changes the imaging parameter to the SAR limit value and performs the imaging of the subject 100. As a result, the damage to the receive coil 51A can be suppressed.

Further, it goes without saying that the present invention is not limited to the embodiments described above, and that various modifications are possible.

### EXPLANATION OF REFERENCES

10: medical image diagnostic system
20: MRI apparatus
32: table
34: movable top plate
36: movement rail
41: space
50, 50A, 50B, 50C, 50D, 50E, 50F: receive coil unit
51, 51A, 51B: receive coil
52: coil element
55: plate-shaped member
56: coil cover
57: body part
60: guide band
61: coil connector
62: band hook
64: handle
66: buckle
68: rail runner
76: mounting sensor
77: length sensor
78: transmission line
100: subject

What is claimed is:

1. A receive coil unit comprising:
a band member;
a first connecting tool and a second connecting tool that are provided at both ends of the band member and connect the band member to a table;
a receive coil that includes a plurality of coil elements receiving a nuclear magnetic resonance signal of a subject; and
a hook member that is disposed on the receive coil and holds the band member so that the receive coil is slidable along the band member.

2. The receive coil unit according to claim 1,
wherein the receive coil does not have the hook members at both ends and includes weight members at the both ends.

3. The receive coil unit according to claim 1,
wherein the receive coil includes the plurality of coil elements and a plurality of plate-shaped members that support the plurality of coil elements and are disposed to be spaced apart from each other,
the plurality of plate-shaped members are configured to be folded, and
the hook member is disposed to be spaced apart from the plurality of plate-shaped members.

4. The receive coil unit according to claim 1,
wherein the receive coil includes a flexible coil cover that covers a periphery of the plurality of coil elements.

5. The receive coil unit according to claim 1,
wherein the receive coil includes the plurality of coil elements and a rigid body portion that holds the plurality of coil elements.

6. The receive coil unit according to claim 1,
wherein the first connecting tool includes a length adjustment mechanism of the band member and a tightening-up mechanism.

7. The receive coil unit according to claim 1,
wherein the first connecting tool includes a length measurement sensor for measuring a length of the band member and/or a connection confirmation sensor for confirming presence or absence of connection of the first connecting tool, and a transmission line that transmits a detection signal from the length measurement sensor and/or the connection confirmation sensor to the receive coil.

8. A medical image diagnostic system comprising:
the receive coil unit according to claim 1;
a table including a movable top plate on which a subject is placed; and
a magnetic resonance imaging apparatus including a processor that processes a nuclear magnetic resonance signal received by the receive coil unit.

9. The medical image diagnostic system according to claim 8,
wherein the movable top plate includes a movement rail along a longitudinal direction, to which the second connecting tool is slidably connected.

10. The medical image diagnostic system according to claim 9,
wherein the table includes a retracting rail for retracting the receive coil unit.

11. The medical image diagnostic system according to claim 10,
wherein the receive coil unit includes a coil connector, and
the table includes an accommodation space that accommodates the coil connector.

12. A medical image diagnostic system comprising:
the receive coil unit according to claim 1;
a length measurement sensor for measuring a length of the band member provided in the first connecting tool, a connection confirmation sensor for confirming presence or absence of connection of the first connecting tool, and a transmission line for transmitting a detection signal from the length measurement sensor and the connection confirmation sensor to the receive coil; and
a magnetic resonance imaging apparatus including a processor and an input receiving device that process a nuclear magnetic resonance signal received by the receive coil unit,
wherein the processor is configured to determine an SAR limit value on the basis of the detection signal from the length measurement sensor, and display the SAR limit value on the input receiving device, and the input receiving device is configured to receive a change of an imaging parameter.
